(19) United States
(12) Patent Application Publication
(10) Pub. No.: US 2023/0172430 A1
(43) Pub. Date: Jun. 8, 2023

(54) DEVICES, SYSTEMS, AND METHODS FOR ENDOSCOPIC VALVES

(71) Applicant: Boston Scientific Scimed, Inc., Maple Grove, MN (US)

(72) Inventors: Ryan V. Wales, Northborough, MA (US); Brian Luis, Boylston, MA (US)

(73) Assignee: Boston Scientific Scimed, Inc., Maple Grove, MN (US)

(21) Appl. No.: 17/992,534

(22) Filed: Nov. 22, 2022

Related U.S. Application Data
(60) Provisional application No. 63/285,231, filed on Dec. 2, 2021.

(51) Int. Cl. A61B 1/00; A61B 1/015

(52) U.S. Cl. CPC A61B 1/00068; A61B 1/015; A61B 1/00103

(57) ABSTRACT

Various embodiments are generally directed to devices, systems, and methods for controlling the flow of fluids in endoscopic systems, such as endoscopic ultrasound (EUS) enabled endoscopes. Some embodiments are particularly directed to valve sets and/or valve interface mechanisms for controlling air and water flow through a valve well for an endoscopic system. Several embodiments are directed to user interface mechanisms and techniques for enabling an operator to interact with and control endoscope valves. In one or more embodiments, manufacture of the valve sets and/or valve interface mechanisms, collectively referred to as flow controllers, may be simplified, such as to make it economical to dispose of the flow controller after a single use.

### CROSS REFERENCE TO RELATED APPLICATIONS

This application claims the benefit of U.S. Provisional Patent Application Ser. No. 63/285,231 filed on Dec. 2, 2021, the disclosure of which is incorporated herein by reference.

### FIELD

The present disclosure relates generally to the field of medical devices. In particular, the present disclosure relates to devices, systems, and methods to control flow through a valve well for an endoscope.

### BACKGROUND

An endoscopy procedure is used in medicine to access the interior of a body for diagnostic and/or therapeutic procedures. Oftentimes, the endoscopy procedure uses an endoscope to examine or manipulate the interior of a hollow organ or cavity of the body. Unlike many other medical imaging techniques, endoscopes are inserted directly into the organ. Typically, an endoscope includes one or more channels for the flow of one or more fluids therethrough. For example, air and water may selectively flow through an endoscope. A valve assembly may be configured and used in various fashion to control the flow of the air and water through the endoscope. In the case of some endoscopes, such as an echoendoscope or ultrasound endoscope, the control of fluids may also be used to inflate and deflate a balloon at the end of an endoscope.

It is with these considerations in mind that a variety of advantageous outcomes may be realized by the devices, systems, and methods of the present disclosure

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In one aspect, the present disclosure relates to a medical device, comprising a primary control valve and one or more seals. The primary control valve may include a top, a bottom, one or more orifices, and a lumen in fluid communication with at least one orifice in the one or more orifices. The one or more seals may be positioned between the top and bottom of the primary control valve. The primary control valve and the one or more seals may comprise a unitary structure.

In some embodiments, the primary control valve and the one or more seals are formed from an elastomer. In some such embodiments, the elastomer comprises nitrile rubber. In various such embodiments, the primary control valve comprises a first Shore value and the one or more seals comprise a second Shore value lower than the first Shore value. In many such embodiments, the first Shore value is a Shore A value between 50 and 100. In one such embodiment, the second Shore value is a Shore A value between 5 and 50. Some embodiments include a housing configured to couple with the primary control valve and a valve well. In some such embodiments, the housing is configured to provide a biasing force between the primary control valve and the valve well. In various such embodiments, the housing is formed from an elastomer. Many embodiment include a variable rate spring disposed about a portion of the primary control valve. In many such embodiments, the portion of the primary control valve is between the top of the primary control valve and the one or more seals. In several embodiments, the one or more seals are overmolded onto the primary control valve to form the unitary structure. In various embodiments, the primary control valve is formed in a first stage of a molding process and the one or more seals are formed in a second stage of the molding process. In various such embodiments, the first stage of the molding process utilizes injection molding. In many such embodiments, the second stage of the molding process utilizes injection molding and overmolding.

In another aspect, the present disclosure relates to a method. The method may include forming a primary control valve in a first stage of a molding process and forming one or more seals on the primary control valve in a second stage of the molding process to create a unitary structure having the primary control valve and the one or more seals.

In various embodiments, the method includes allowing the primary control valve to begin to set between the first and second stages of the molding process. In some embodiments, the primary control valve and the one or more seals are formed from an elastomer. In some such embodiments, the elastomer comprises nitrile rubber. In many embodiments, the molding process comprises an injection molding process. In several embodiments, the molding process comprises an overmolding process.

In yet another aspect, the present disclosure relates to a medical device, comprising a primary control valve, one or more seals, and a housing. The primary control valve may include a top, a bottom, one or more orifices, and a lumen in fluid communication with at least one orifice in the one or more orifices. The one or more seals may be positioned between the top and bottom of the primary control valve. The primary control valve and the one or more seals may comprise a unitary structure. The housing may be configured to couple with the primary control valve and a valve well.

In some embodiments, the housing is configured to provide a biasing force between the primary control valve and the valve well. In many embodiments, the housing is formed from an elastomer. Various embodiments include a variable rate spring disposed about a portion of the primary control valve. In several embodiments, the one or more seals are overmolded onto the primary control valve to form the unitary structure.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. In will be appreciated that various figures included in this disclosure may omit some components, illustrate portions of some components, and/or present some components as transparent to facilitate illustration and description of components that may otherwise appear hidden. For purposes of clarity, not every component is labeled in every figure, nor is every component of each embodiment shown where illustration is not necessary to allow those of ordinary skill in the art to understand the disclosure. In the figures:

FIG. 1 a block diagram of an air/water (AW) valve assembly according to one or more embodiments described hereby.

FIGS. 2A-2E illustrate various aspects of an exemplary AW valve well according to one or more embodiments described hereby.

FIG. 3 illustrates an exemplary AW valve set according to one or more embodiments described hereby.

FIGS. 4A-6C illustrate various aspects of exemplary valves in AW valve sets according to one or more embodiments described hereby.

FIG. 7 illustrates an exemplary AW valve well according to one or more embodiments described hereby.

FIGS. 8A and 8B illustrate an exemplary flow controller according to one or more embodiments described hereby.

FIG. 9 illustrates a cross-sectional view of an exemplary flow controller according to one or more embodiments described hereby.

FIGS. 10A and 10B illustrate various aspects of a flow controller according to one or more embodiments described hereby.

FIG. 10C illustrates an aspect of the subject matter in accordance with one embodiment.

### DETAILED DESCRIPTION

Various embodiments are generally directed to devices, systems, and methods for controlling the flow of fluids in endoscopic systems, such as endoscopic ultrasound (EUS) enabled endoscopes. Some embodiments are particularly directed to valve sets and/or valve interface mechanisms for controlling air and water flow through a valve well for an endoscopic system. Several embodiments are directed to user interface mechanisms and techniques for enabling an operator to interact with and control endoscope valves. In one or more embodiments, manufacture of the valve sets and/or valve interface mechanisms, collectively referred to as flow controllers, may be simplified, such as to make it economical to dispose of the flow controller after a single use. These and other embodiments are described and claimed.

Some challenges when controlling the flow of fluids through endoscopes include unreliable valves prone to failure. For example, many flow controllers are fragile and likely to leak. These issues can be compounded when the components are designed, constructed, and/or assembled economically to facilitate disposal after a single use. Alternatively, these issues can be compounded when reusable components are worn down from multiple use/cleaning cycles. Adding further complexity, existing flow controllers may be assembled from an excessive number of components, assemblies, and/or steps. For example, some flow controller may be assembled from over 11 components which feed into 6 distinctive assemblies that are combined to complete the flow controller. This complexity drives up the cost to manufacture each flow controller. These and other factors may result in devices, systems, and methods for controlling the flow of fluids through endoscopes that are difficult to use, expensive, and inefficient, resulting in limited applicability and/or uncertain outcomes. Such limitations can drastically reduce the dependability, disposability, and intuitiveness of flow control in endoscopes and procedures performed therewith, contributing to reduced usability, adverse outcomes, excess fatigue, and lost revenues.

Various embodiments described hereby include one or more components of a flow controller, such as valves and/or valve interface mechanisms, that provide reliable and economical control of fluid flow through endoscopes. In several embodiments, the components may provide reliable operation while providing sufficient value to be disposable (e.g., single-use). For example, sufficient value to be disposable may be realized by reducing the complexity of manufacturing the flow controllers, such as through a reduction in the number of components, assemblies, and/or steps to manufacture the flow controller. In many embodiments, the components may provide accurate and intuitive interfaces to improve operator experience, such as via tactile feedback. For example, one or more components of the valve interface mechanism may provide tactile or haptic feedback to indicate how a set of valves is arranged (e.g., arranged to permit/block flows between various channels). In some examples, the force to operate a user interface mechanism may vary, such as due to a variable pitch spring, to indicate transitions between valve states. In various embodiments, tactile feedback may be produced as a result of different components of a valve assembly coming into contact, such as due to received input.

In various embodiments, one or more of the components may be designed to simplify manufacturability. For instance, the location or construction of one or more biasing members may simplify component assembly. In another instance, multiple components may be integrally formed to simplify component assembly. In these and other ways, components/techniques described here may improve operator experience, decrease learning curves, improve reliability, and/or decrease manufacturing complexity via realization of more efficient and valuable devices, systems, and methods for controlling the flow of fluids in endoscopic systems. In many embodiments, one or more of the advantageous features may result in several technical effects and advantages over conventional technology, including increased capabilities and improved adaptability.

The present disclosure is not limited to the particular embodiments described. The terminology used herein is for the purpose of describing particular embodiments only and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure may be described with specific reference to specific medical devices and systems (e.g., an endoscope), it should be appreciated that such medical devices and systems may be used in a variety of medical procedures which require navigating one or more accessory tools through ductal, luminal, or vascular anatomies, including, for example, interventional radiology procedures, balloon angioplasty procedures, thrombolysis procedures, angiography procedures, Endoscopic Retrograde Cholangio-Pancreatography (ERCP) procedures, and the like. The disclosed medical devices and systems may be inserted via different access points and approaches, e.g., percutaneously, endoscopically, laparoscopically or some combination thereof.

As used herein, a “proximal” end refers to the end of a device that lies closest to the medical professional along the device when introducing the device into a patient, and a “distal” end refers to the end of a device or object that lies furthest from the medical professional along the device during implantation, positioning, or delivery.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etcetera, indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified. The recitation of numerical ranges by endpoints includes all numbers within that range, including the endpoints (e.g., 1 to 5 includes 1, 1.5, 2, 2.75, 3, 3.80, 4, and 5).

As used herein, the conjunction “and” includes each of the structures, components, portions, or the like, which are so conjoined, unless the context clearly indicates otherwise, and the conjunction “or” includes one or the others of the structures, components, portions, or the like, which are so conjoined, singly and in any combination and number, unless the context clearly indicates otherwise.

The detailed description should be read with reference to the drawings, which are not necessarily to scale, depict illustrative embodiments, and are not intended to limit the scope of the invention.

Reference is now made to the drawings, wherein like reference numerals are used to refer to like elements throughout. In the following description, for purpose of explanation, numerous specific details are set forth in order to provide a thorough understanding thereof. It may be evident, however, that the novel embodiments can be practiced without these specific details. In other instances, well known structures and devices are shown in block diagram form to facilitate a description thereof. The intention is to cover all modification, equivalents, and alternatives within the scope of the claims.

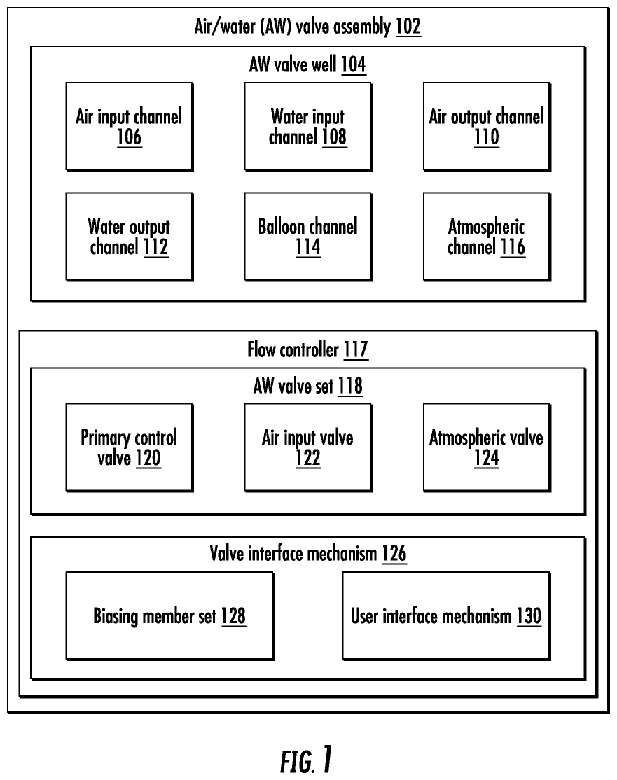

FIG. 1 illustrates a block diagram of an air/water (AW) valve assembly 102, according to one or more embodiments described hereby. In some embodiments, one or more components of AW valve assembly 102 may be the same or similar to one or more other components described hereby. AW valve assembly 102 includes an AW valve well 104 and a flow controller 117 comprising an AW valve set 118 and a valve interface mechanism 126. In one or more embodiments described hereby, various components of AW valve assembly 102 may interoperate to provide reliable and intuitive control of fluid flow through endoscopic systems. For example, one or more components of valve set 118 and valve interface mechanism 126 may provide reliable and intuitive control of fluid flow through AW valve well 104. In several embodiments, the AW valve set 118 and the valve interface mechanism 126 may be assembled together and then coupled to AW valve well 104 to form AW valve assembly 102. In many embodiments, components of a valve assembly may be classified as, belong to, include, implement, and/or interoperate with one or more of a valve well, a valve set, and a valve interface mechanism. For instance, a valve interface mechanism may include one or more portions of a valve. Embodiments are not limited in this context.

In several embodiments, the AW valve well 104 may include air input channel 106, water input channel 108, air output channel 110, water output channel 112, balloon channel 114, and atmospheric channel 116; the AW valve set 118 may include primary control valve 120, air input valve 122, and atmospheric valve 124; and the valve interface mechanism 126 may include biasing member set 128 and user interface mechanism 130. In various embodiments, the channels of the AW well 104 may be connected to other components in an endoscopic system, such as via tubing or piping. In one or more embodiments described hereby, the air input channel 106 may be connected to a pressurized air source, the water input channel 108 may be connected to a water source, the air output channel 110 may be connected to an air channel of an endoscopic device (e.g., endoscope or component disposed therethrough), the water output channel 112 may be connected to a water channel of an endoscopic device, and the balloon channel 114 may be connected to a balloon of an endoscopic device. In several embodiments, AW valve set 118 and valve interface mechanism 126 may control the flow of air and water through AW valve well 104. In several such embodiments, the flow of air may be controlled from air input channel 106 to one of the air output channel 110, the atmospheric channel 116, or blocked, and/or the flow of water may be controlled from water input channel 108 to one of water output channel 112, the balloon channel 114, or blocked.

In many embodiments, AW valve assembly 102 may be used in conjunction with an endoscopic system, such as an EUS system. In various embodiments, reference to a balloon may refer to a balloon in the EUS system that can be inflated/deflated to provide medium to facilitate transmission of sound waves and capturing of ultrasound images. For example, valve interface mechanism 126 may receive input to control the flow of water through AW valve well to inflate a balloon by arranging the AW valve set 118 to place the water input channel 108 in fluid communication with balloon channel 114. In other embodiments, one or more of the components of the AW valve assembly may be implemented in configurations that do not require or include a balloon, such as a video capable scope with ultrasound functionality.

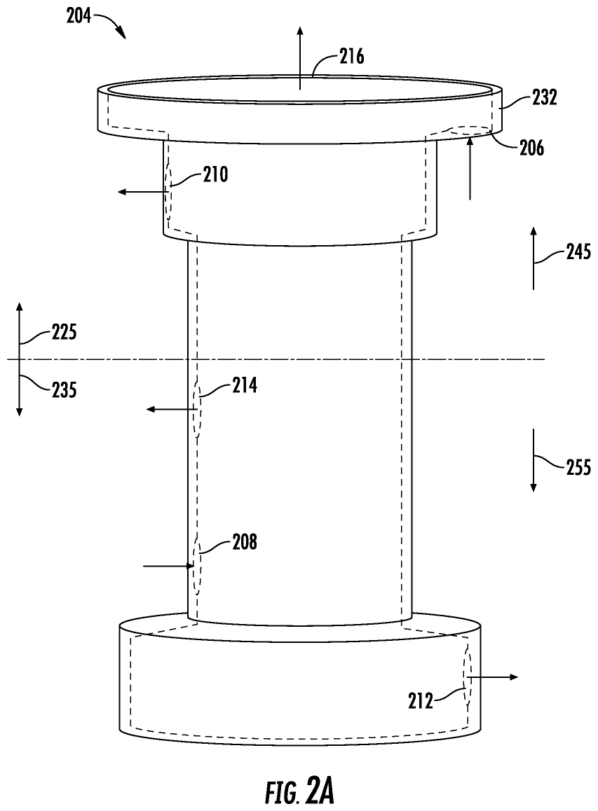

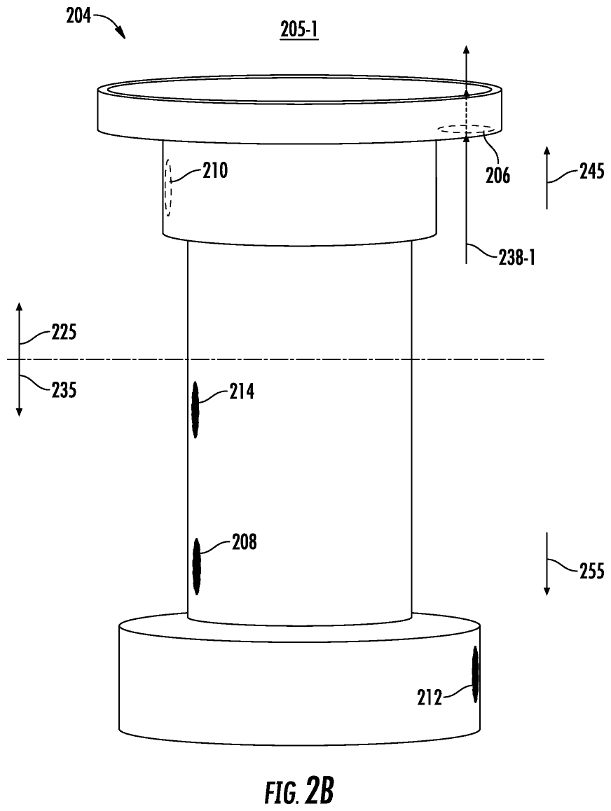

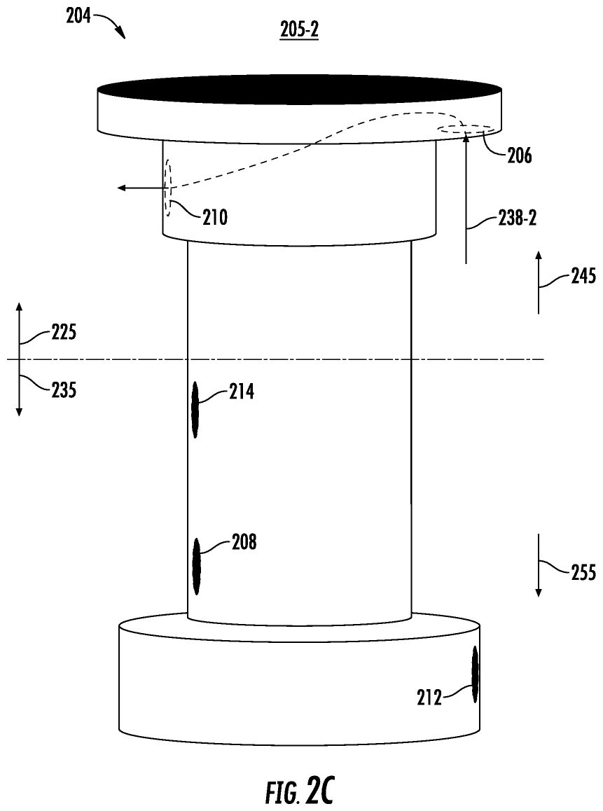

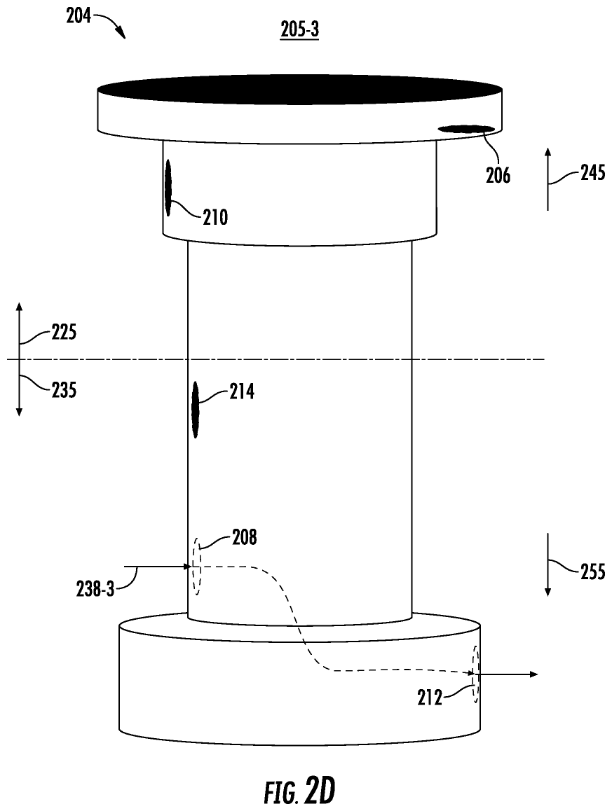

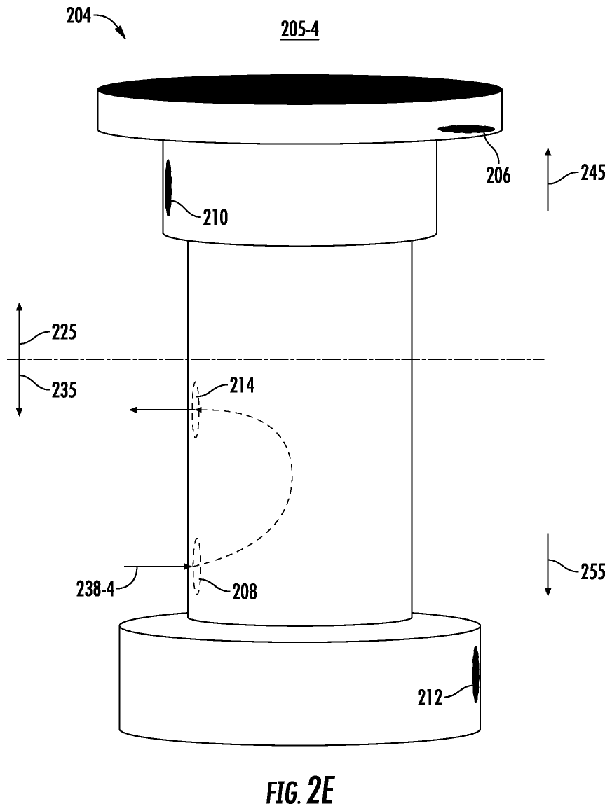

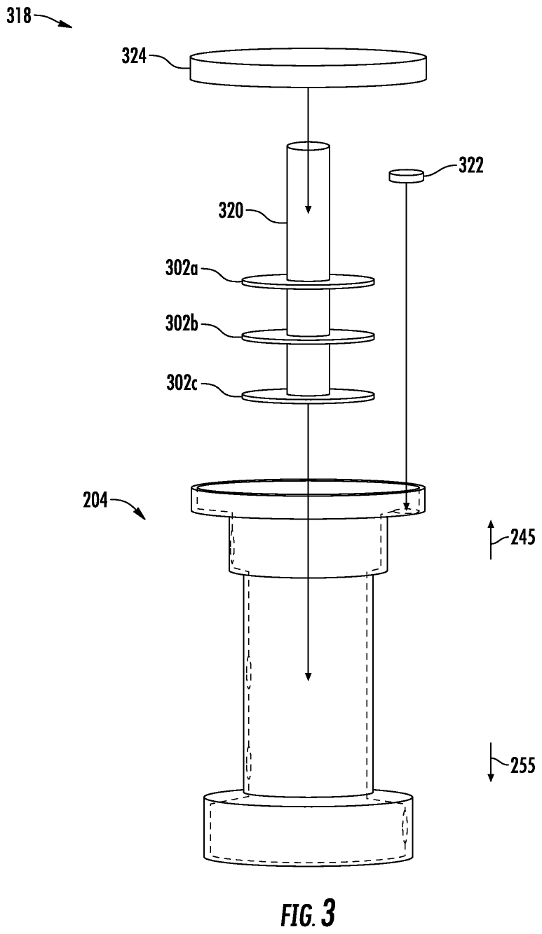

More generally, in several embodiments, each channel in a valve well may refer to a flow path comprising an input/output of a fluid from/to a corresponding entity. For example, atmospheric channel 116 may refer to a flow path comprising an output to the atmosphere. These and other aspects of the present disclosure will be described in more detail below, such as with respect to FIGS. 2A-2E. In various embodiments, each valve in a valve set may refer to a component that physically controls flow through or between one or more channels. For instance, when closed, the atmospheric valve 124 may block the flow of air out of the atmospheric channel 116. In another instance, in a first position, or first state, the primary control valve 120 may place the water input channel 108 in fluid communication with the water output channel 112, and in a second position, the primary control valve 120 may place the water input channel 108 in fluid communication with the balloon channel 114. These and other aspects of the present disclosure will be described in more detail below, such as with respect to FIGS. 3-6C.

In various embodiments, the valve interface mechanisms may include one or more components to enable control over the arrangement of valves in a valve set. In such embodiments, biasing member sets may include one or more, torsional springs, lever springs, coil spring, baffles, dampers, clips, and the like that provide a force to bias one or more components in a specific direction or position. For example, the biasing member set 128 may cause air to flow out the atmospheric channel when no input is being received. In an additional, or alternative example, the biasing member set 128 may provide differing resistance to operation of the user interface mechanism 130 between different states, such as to provide tactile indications of the state. In various embodiments, the user interface mechanism 130 may include one or more of an interface, an interface member, a user interface, a housing, a linkage, a knob, a lever, a rocker switch, a push/pull switch, a knob, a button, a diaphragm switch, a toggle switch, and the like. In some embodiments, an interface, an interface member, and/or a user interface may be the same or similar.

In several embodiments, user interface mechanisms may include one or more components to receive input and/or implement valve arrangements. For example, user interface mechanism 130 may include a user interface comprising a lever and one or more linkages to translate motion of the lever into appropriate motion of one or more valves to achieve a desired flow. In various embodiments, user interface mechanisms may include one or more biasing members and/or biasing members may include one or more user interface mechanisms. It will be appreciated that one or more components described hereby in the context of an AW valve assembly may be adapted for use in other valve assemblies (e.g., a suction valve assembly), and vice versa, without departing from the scope of this disclosure. These and other aspects of the present disclosure will be described in more detail below.

FIGS. 2A-2E illustrate various aspects of an AW valve well 204, according to one or more embodiments described hereby. In some embodiments, one or more components of FIGS. 2A-2E may be the same or similar to one or more other components described hereby. AW valve well 204 includes an air input channel 206, a water input channel 208, an air output channel 210, a water output channel 212, a balloon channel 214, and an atmospheric channel 216. In one or more embodiments described hereby, fluid may flow through the valve wells based on the arrangement of one or more valves as positioned by one or more valve interface mechanisms. Embodiments are not limited in this context.

Referring to FIG. 2A, the AW valve well 204 may include a top 245 and a bottom 255 and/or an air portion 225 and a water portion 235. The air output channel 210, air input channel 206, and atmospheric channel 216 may be in the air portion 225. The atmospheric channel 216 may comprise a horizontally-oriented exit towards the top 245 and lip 232, the air input channel 206 may comprise a horizontally-oriented entrance towards the top 245, the air output channel 210 may comprise a vertically-oriented exit towards the top. The water input channel 208, water output channel 212, and balloon channel 214 may be in the water portion 235. The balloon channel 214 may comprise a vertically-oriented exit proximate the middle, the water input channel 208 may comprise a vertically-oriented entrance toward the bottom 255, and the water output channel 212 may comprise a vertically-oriented exit toward the bottom 255. In several embodiments, the lip 232 may enable one or more suction valve sets and/or valve interface mechanisms to couple to the AW valve well 204.

In several embodiments, the AW valve well 204 may change diameters one or more times. For example, the diameter changes in conjunction with vertical displacement of a valve may enable flow around the valve and through a channel. In the illustrated embodiment, the AW valve well may have a first diameter comprising the entrance/exit of the respective air input and atmospheric channels 206, 216, a second diameter comprising the exit of the air output channel 210, a third diameter comprising the entrance/exit of the respective water input and balloon channels 208, 214, and a fourth diameter comprising the exit of the water output channel 212. It will be appreciated that the orientation, size, and/or arrangement of one or more of the channels and/or flows may be modified in various embodiments without departing from the scope of this disclosure.

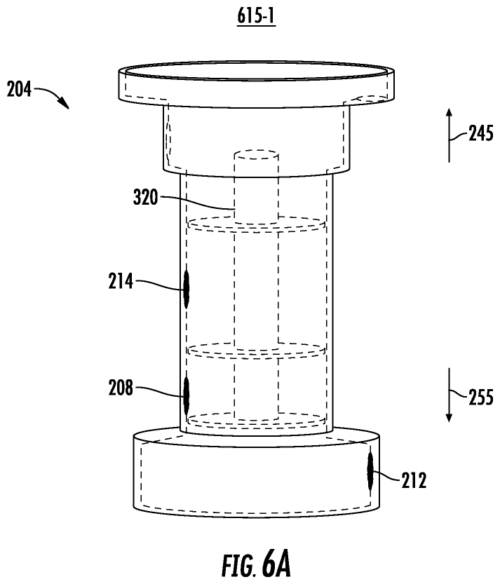

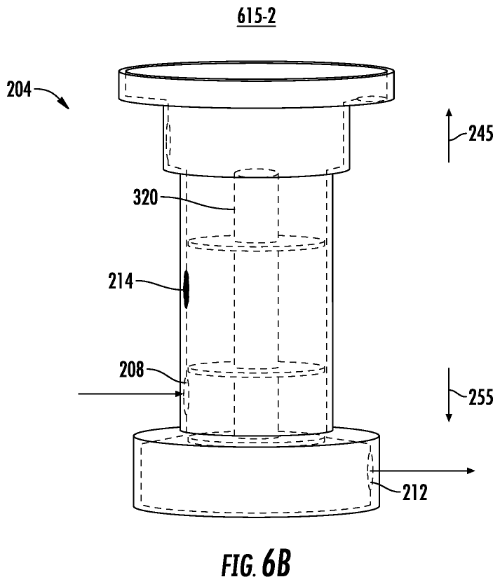

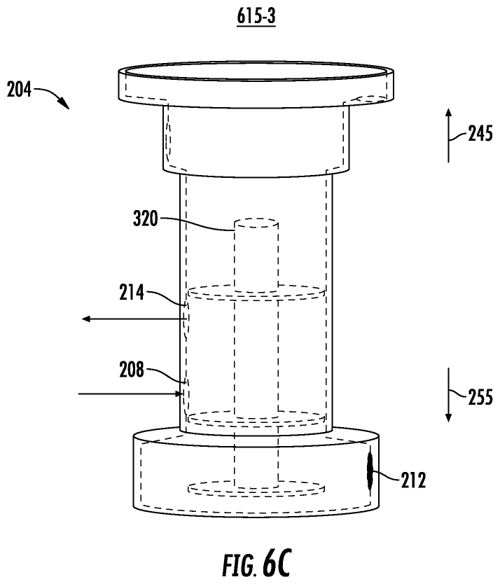

FIG. 2B illustrates a flow 238-1 through the AW valve well 204 in an air escape state 205-1. In the air escape state 205-1, flow 238-1 may enter via air input channel 206 and exit through the atmospheric channel 216. Further, in some embodiments, flow may be blocked through one or more of balloon channel 214, water input channel 208, and water output channel 212. In FIGS. 2B-2E, an opening that is filled with black indicates that there is no net flow through that opening. Accordingly, in the air escape state 205-1 of FIG. 2B, there is no net flow through the balloon channel 214, the water input channel 208, and the water output channel 212 (see e.g., FIGS. 6A-6C).

FIG. 2C illustrates a flow 238-2 through the AW valve well 204 in an air delivery state 205-2. In the air delivery state 205-2, flow 238-2 may enter via the air input channel 206 and exit through the air output channel 210. Further, in various embodiments, flow may be blocked through one or more of atmospheric channel 216, balloon channel 214, water input channel 208, and water output channel 212.

FIG. 2D illustrates a flow 238-3 through the AW valve well 204 in a water delivery state 205-3. In the water delivery state 205-3, flow 238-3 may enter via water input channel 208 and exit through the water output channel 212. Further, in various embodiments, flow may be blocked through one or more of the balloon channel 214, air output channel 210, air input channel 206, and atmospheric channel 216. In various embodiments, blocking flow at the air input channel 206 may cause pressure to build in a water source feeding the water input channel 208. In various such embodiments, pressure in the water source may cause fluid to flow from the water source to water input channel 208.

FIG. 2E illustrates a flow 238-4 through the AW valve well 204 in a balloon fill state 205-4. In the balloon fill state 205-4, flow 238-4 may enter via the water input channel 208 and exit through the balloon channel 214. Further, in many embodiments, flow may be blocked through one or more of the water output channel 212, air output channel 210, air input channel 206, and atmospheric channel 216.

FIGS. 3-6C illustrate various aspects of exemplary valves for AW valve wells, according to one or more embodiments described herein. More specifically, FIGS. 3-6C illustrate various aspects of an AW valve set 318 in conjunction with one or more components of AW valve well 204. In one or more embodiments described herein, fluid may flow through the valve wells based on the arrangement of one or more valves as positioned by one or more valve interface mechanisms. In many embodiments, one or more valves described herein may include a plurality of components configured to control fluid through a valve well. In some embodiments, one or more components of FIGS. 3-6C may be the same or similar to one or more other components described herein. Embodiments are not limited in this context.

FIG. 3 illustrates AW valve set 318 in conjunction with AW valve well 204. AW valve set 318 may include primary control valve 320 with seals 302a, 302b, 302c, air input valve 322, and atmospheric valve 324. In several embodiments, the primary control valve 320 may be inserted into the AW valve well 204 to control, at least in part, the flow through one or more channels of the AW valve well 204. In various embodiments, the air input valve 322 may be inserted into the air input channel of the AW valve well 204 to control flow therethrough. In many embodiments, the atmospheric valve 324 may be inserted into the atmospheric channel of AW valve well 204 to control flow therethrough. In many embodiments, one or more valves in AW valve set 318 may be integrated with one or more portions of a housing and/or valve interface mechanism corresponding to AW valve well 204.

In one or more embodiments, the atmospheric valve 324 may be configured to control fluid communication with the atmosphere from the interior of the AW valve well 204. In many embodiments, the atmospheric valve 324 may include a hole in a housing. In some embodiments, the atmospheric valve 324 may be operated by covering and/or uncovering the hole, such as with a finger or other mechanism. In several embodiments, the positioning and/or configuration of the valves in AW valve set 318 may be controlled by one or more components of a corresponding valve interface mechanism. In some embodiments, one or more portions of the atmospheric channel 216 may be included in the primary control valve 320. In some such embodiments, the atmospheric channel 216 may comprise one or more passages through at least a portion of the primary control valve 320. For example, the atmospheric channel 216 may comprise a hole in the top of the primary control valve 320 in fluid communication with a radial hole in the primary control valve 320 proximate the air input channel 206. In such examples, covering the hole may direct air flow into the air output channel 10 and down a working channel of an endoscope. In many embodiments, as will be described in more detail below, positioning of the seals 302a, 302b, 302c of primary control valve 320 by moving the primary control valve 320 up and down may be utilized to control the flow of fluid through the water portion 235 of the AW valve well 204.

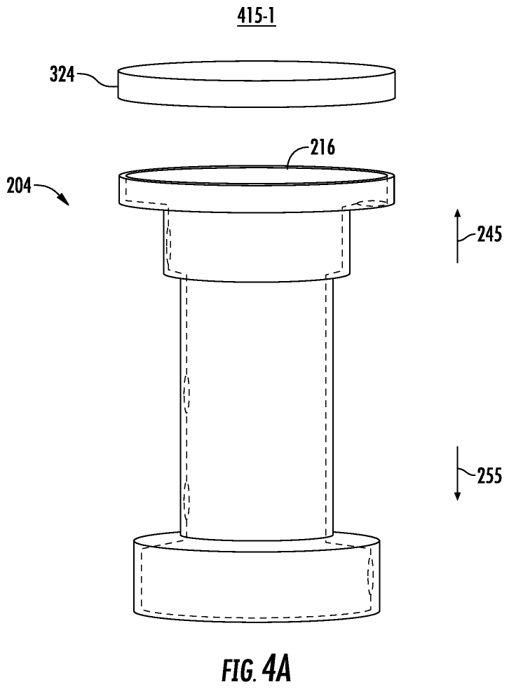

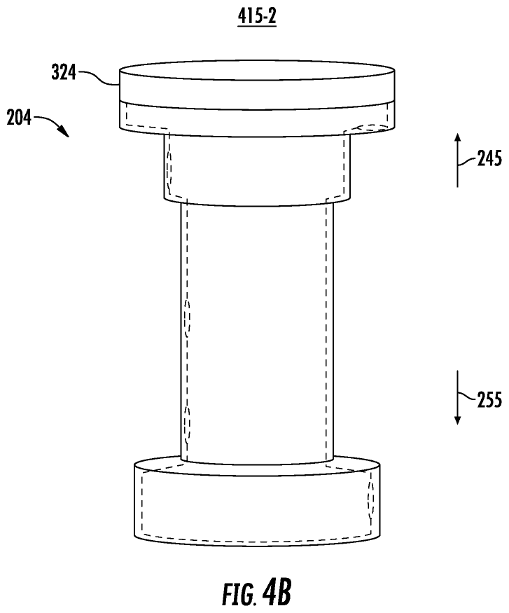

FIG. 4A illustrates an atmospheric valve open state 415-1. In the atmospheric valve open state 415-1, the atmospheric valve 324 may allow flow through the atmospheric channel of AW valve well 204.

FIG. 4B illustrates an atmospheric valve sealed state 415-2. In the atmospheric valve sealed state 415-2, the atmospheric valve 324 may prevent flow through atmospheric channel of AW valve well 204. As will be discussed in more detail below, in operation, fluid communication with the atmosphere may be provided through a passage/channel in, or created by, one or more components (e.g., primary control valve 320). Further, one or more components may be used to seal portions of the atmospheric channel 216 to facilitate control of fluid communication with the atmosphere by atmospheric valve 324. In some embodiments, atmospheric valve 324 may include a plurality of components configured to control fluid communication with the atmosphere.

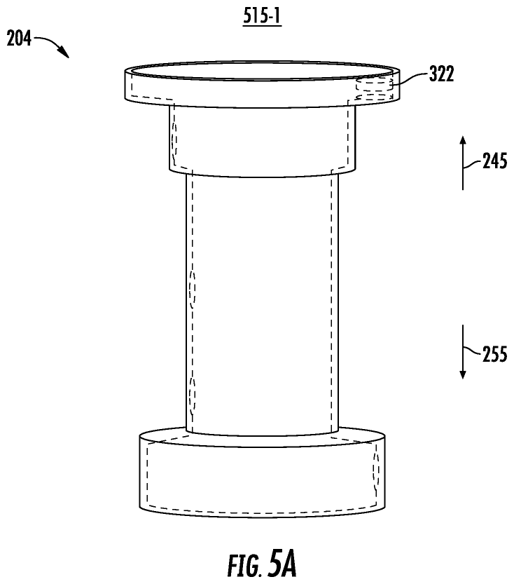

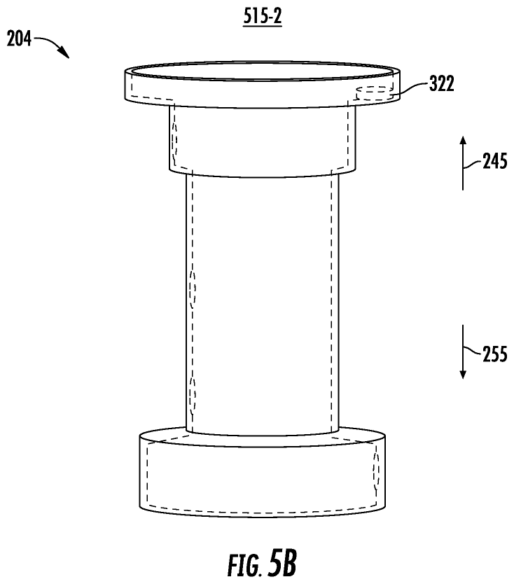

FIG. 5A illustrates an air input valve open state 515-1. In the air input valve open state 515-1, the air input valve 322 may allow flow through the air input channel of AW valve well 204.

FIG. 5B illustrates an air input valve sealed state 515-2. In the air input valve sealed state 515-2, the air input valve 322 may prevent flow through the air input channel of AW valve well 204. In some embodiments, sealing the air input channel may cause a fluid source (e.g., water reservoir) to be pressurized, thereby enabling/causing fluid to flow into the AW valve well 204 via water input channel 208.

FIG. 6A illustrates a primary valve sealed state 615-1. In the primary valve sealed state 615-1, the primary control valve 320 may prevent flow through one or more of the balloon channel 214, water input channel 208, and water output channel 212.

Referring to FIG. 6B illustrates a primary valve water output state 615-2. In the primary valve water output state 615-2, the primary control valve 320 may be positioned to block flow through balloon channel 214 and permit flow from water input channel 208 to water output channel 212. In various embodiments, primary control valve 320 may utilize changes in diameter in AW valve well 204 to control flow.

FIG. 6C illustrates a primary valve balloon fill state 615-3. In the primary valve balloon fill state 615-3, the primary control valve 320 may be positioned to block flow through water output channel 212 and permit flow from water input channel 208 to balloon channel 214. In various embodiments, one or more features of primary control valve 320 may operate as valves for multiple channels. In some embodiments, one or more features of primary control valve 320 may comprise one or more channels, or one or more portions thereof. For example, primary control valve 320 may comprise atmospheric channel 216.

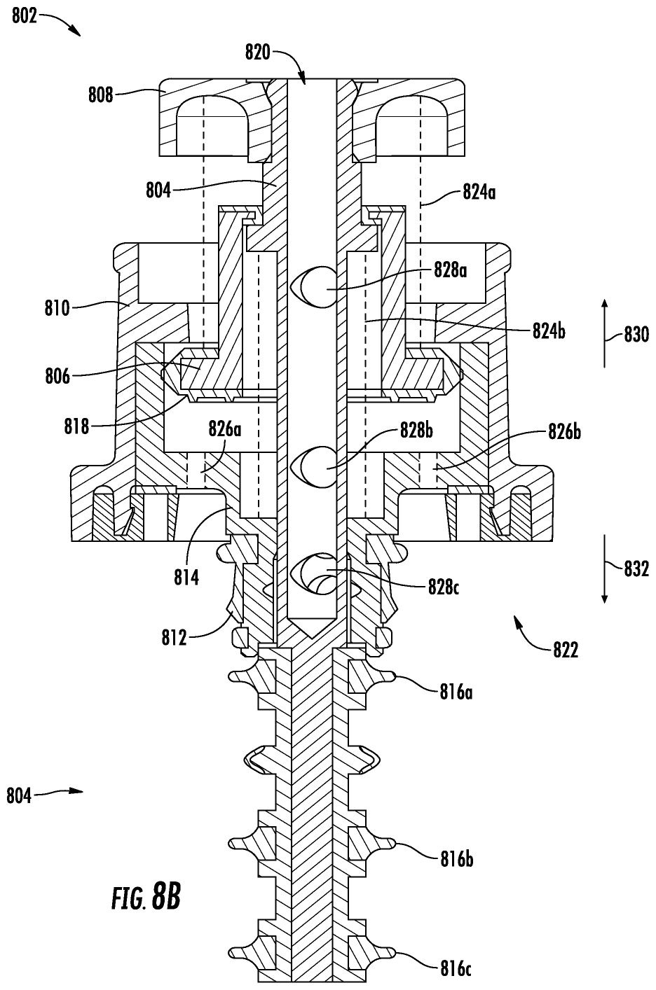

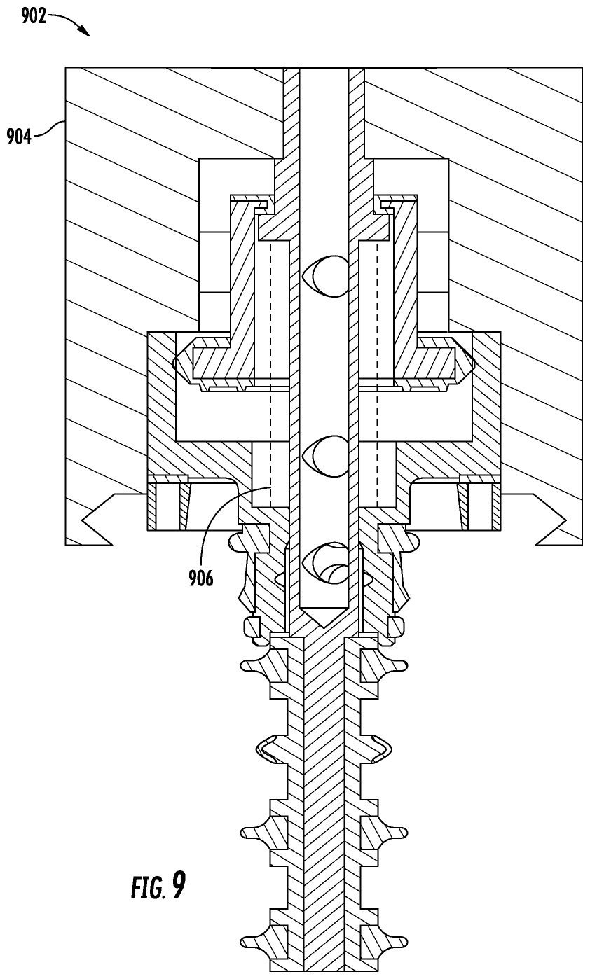

FIG. 7 illustrates an AW valve well 702 according to one or more embodiments disclosed hereby. The AW valve well 702 may include a water output channel 704, an air output channel 706, an air input channel 708, a balloon channel 710, a water input channel 712, and a housing interface 714. In various embodiments, the housing interface 714 may couple with one or more components of a flow controller (e.g., flow controller 802, 902, or 1002). In some embodiments, FIG. 7 may include one or more components that are the same or similar to one or more other components of the present disclosure. For example, AW valve well 702 may be the same or similar to AW valve well 204. Further, one or more components of FIG. 7, or aspects thereof, may be incorporated into other embodiments of the present disclosure, or excluded from the disclosed embodiments, without departing from the scope of this disclosure. For example, balloon channel 710 may be excluded from AW valve well 702 without departing from the scope of this disclosure. Still further, one or more components of other embodiments of the present disclosure, or aspects thereof, may be incorporated into one or more components of FIG. 7, without departing from the scope of this disclosure. For example, flow controller 902 of FIG. 9 be coupled to AW valve well 702 to form an AW valve assembly without departing from the scope of this disclosure. Embodiments are not limited in this context.

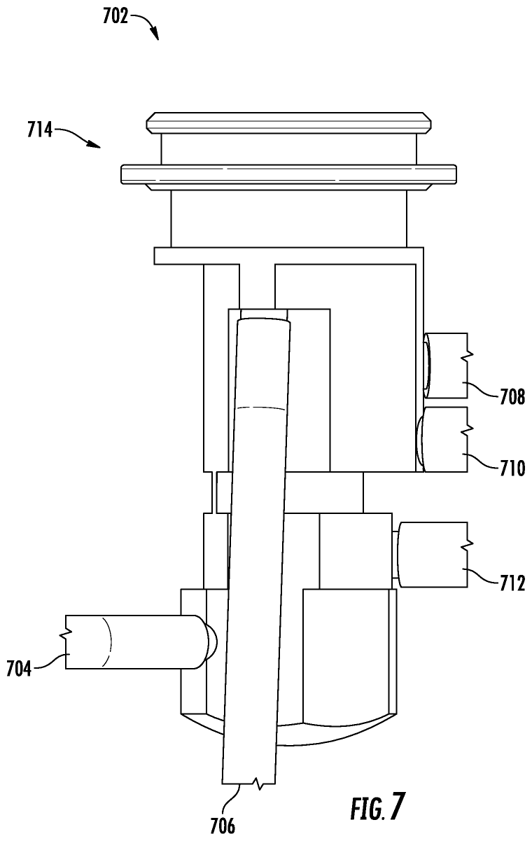

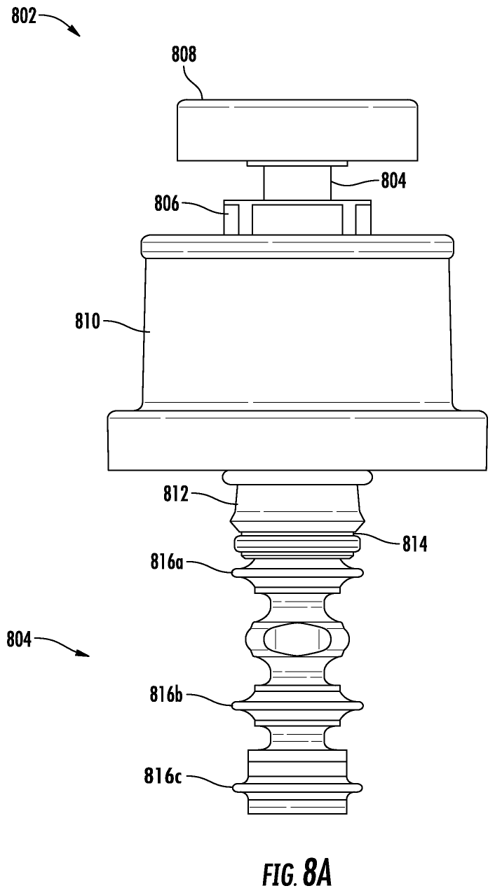

FIGS. 8A and 8B illustrate a flow controller 802 according to one or more embodiments disclosed hereby. More specifically, FIG. 8A illustrates a front view of flow controller 802 and FIG. 8B illustrates a cross-sectional view of flow controller 802 with a top 830 and a bottom 832. The flow controller 802 may include primary control valve 804, hat 806, interface 808, housing 810, bowl 814, and biasing members 824a, 824b. As better shown in FIG. 8B, primary control valve 804 include seals 816a, 816b, 816c, lumen 820, and orifices 828a, 828b, 828c in fluid communication with lumen 820; housing 810 may include valve well interface 822; bowl 814 may include one-way seal 812 and air channels 826a, 826b; and hat 806 may include air input valve 818. In some embodiments, FIGS. 8A and 8B may include one or more components that are the same or similar to one or more other components of the present disclosure. For example, seals 816a, 816b, 816c of primary control valve 804 may be the same or similar to seals 302a, 302b, 302c of primary control valve 320. Further, one or more components of FIGS. 8A and 8B, or aspects thereof, may be incorporated into other embodiments of the present disclosure, or excluded from the disclosed embodiments, without departing from the scope of this disclosure. Still further, one or more components of other embodiments of the present disclosure, or aspects thereof, may be incorporated into one or more components of FIGS. 8A and 8B, without departing from the scope of this disclosure. For example, AW valve well 702 combined with flow controller 802 to form an AW valve assembly without departing from the scope of this disclosure. Embodiments are not limited in this context.

The primary control valve 804 may extend through the bowl 814, housing 810, and hat 806, and couple with interface 808. Referring to FIG. 8B, biasing member 824a may extend between interface 808 and hat 806 and biasing member 824b may extend between hat 806 and bowl 814. In many embodiments, the primary control valve 804 may slide relative to the hat 806, housing 810, and bowl 814. In several of the embodiments described hereby, seals 816a, 816b, 816c may be integrally formed with primary control valve 804. In other words, seals 816a, 816b, 816c and primary control valve 804 may comprise a unitary structure. In various embodiments, air input valve 818 may cover air channels 826a, 826b to seal the air input channel of a valve well. In many embodiments, a plurality of air channels may be radially distributed about bowl 814, such as in alignment with air channels 826a, 826b.

As previously mentioned, flow controller 802 may be coupled to an A/W valve well (e.g., AW valve well 702) to control flow through the AW valve well. For example, valve well interface 822 may couple with housing interface 714. Accordingly, the illustrated state of flow controller 802 corresponds to the air escape state (see e.g., FIG. 2B) resulting from an atmospheric valve open state (see e.g., FIG. 4A), and air input valve open state (see e.g., FIG. 5A), and a primary valve sealed state (see e.g., FIG. 6A). In many embodiments, in the absence of external input, biasing members 824a, 824b may position flow controller 802 in the air escape state. The lumen 820 of the primary control valve 804 may be sealed by placing a finger or other component over the opening at the top of the primary control valve 804 to transition flow controller 802 from the atmospheric valve open state to an atmospheric valve sealed state (see e.g., FIG. 4B), resulting in the air delivery state (see e.g., FIG. 2C). Depressing the primary control valve 804 downward via interface 808 causing hat 806 to contact bowl 814 and air input valve 818 to cover air channels 826a, 826b may transition the flow controller 802 from the air input valve open state to the air input valve closed state (see e.g., FIG. 5B) and transition the primary control valve 804 from the primary valve sealed state to the primary valve water output state (see e.g., FIG. 6B), resulting in the water delivery state (see e.g., FIG. 2D). Depressing the interface 808 further downward such that interface 808 contacts hat 806 and hat 806 contacts bowl 814 may transition the primary control valve 804 of flow controller 802 from the primary valve water output state to the primary valve balloon fill state (see e.g., FIG. 6C), resulting in the balloon fill state (see e.g., FIG. 2E).

Some flow controllers may include assemblies that are comprised of at least two different parts, such as a structural components and a series of one or more seals or O-rings that prevent air or fluid from leaking between different structural components (e.g., primary control valve 804, hat 806, housing 810, bowl 814). Oftentimes, each of the components are manufactured then the seals are assembled post processing to the parts. Accordingly, a structural component combined with one or more seals or O-rings may be referred to as a subassembly. For example, in some embodiments, hat 806 and air input valve 818 may be a subassembly, primary control valve 804 and seals 816a, 816b, 816c may be a subassembly, and/or bowl 814 and one-way seal 812 may be a subassembly.

However, one or more components of flow controllers described hereby may include structural components that are integrally formed with one or more seals or O-rings, such as to reduce manufacturing complexity. For example, seals 816a, 816b, 816c may be integrally formed with primary control valve 804. Integrally forming multiple components can reduce cost and complexity in manufacturing by requiring less assembly steps or setup time. In several embodiments, different materials or the same materials at different densities, durometers, and/or dimensions may be used to achieve different performance features within the same integrally formed part. For example, one or more subassemblies would become a single part with different Shore values depending on the need (e.g., rigidity, strength, flexibility, and the like). Additionally, or alternatively, dimensions of the integrally formed part may be used to achieve different performance features. For example, thinner dimensions combined with a lower Shore value may be utilized for seals and thicker dimensions combined with a higher Shore value may be utilized for structural parts. In various embodiments, a “Shore” may relate to a particular type of hardness measurement testing device (e.g., a Shore durometer), and a specific value may be a measured hardness of a material.

Many embodiments may utilize a polymer or an elastomer for structural components in addition to seals and gaskets. For example, nitrile rubber, or Buna-N, a synthetic petroleum-based rubber material for structural components in addition to seals and gaskets. In some embodiments, nitrile rubber may be made in several different durometers and Shore values, ranging from 40 (pencil eraser) all the way to 90 (shopping-cart wheel). Accordingly, in many embodiments described hereby, one or more of the subassemblies may become a single part with different Shore values depending on the need (e.g., rigidity, strength, flexibility, and the like). For example, the primary control valve 804 may have a Shore A value between 50 and 100 and the seals 816a, 816b, 816c may have a Shore A value between 5 and 50.

In many embodiments, the part is formed in a multi-stage molding process. For instance, the primary control valve 804 may be formed in a first stage of the molding process and the one or more seals 816a, 816b, 816c may be formed in a second stage of the molding process. In one embodiment, the molding process may include stages with injection molding and/or overmolding. For example, the first stage of the molding process may utilize injection molding and the second stage of the molding process may utilize overmolding in combination with injection molding. In some embodiments, the entire part would be injection molded out of nitrile rubber in stages. For example, in a first stage, material with a high Shore value would be injected until the area of the mold that would make up the body of the part is filled (e.g., primary control valve 804). In a second stage, material with a low Shore value would be injected until the part of the mold that makes up the rings and or seals are filled (e.g., seals 816a, 816b, 816c). In several embodiments, by using the same material with different Shore values, performance issues where materials would separate from each other can be avoided. However, since the Shore values are different the body of the part (e.g., primary control valve 804) is still rigid enough to maintain shape and function throughout use while the seal features are flexible enough to prevent air and water from leaking. In the illustrated embodiment, primary control valve 804 may be injection molded and allowed to cool slightly (high durometer material, rigid). Once slightly cooled and beginning to set, a lower durometer material would be injected (low durometer, flexible seal material) to form seals 816a, 816b, 816c.

In several embodiments, the amount of time to cool slightly may include an amount of time for the first stage component (e.g., primary control valve 804) to cool sufficiently to remove it from the mold. However, in such embodiments, after the among of time for the first stage component to cool. In some embodiments, the lower durometer material may be injected as part of an overmolding process. In many embodiments, when removed the entire part appears and behaves like a single homogeneous part with flexible sections behaving like seals.

FIG. 9 illustrates a flow controller 902 according to one or more embodiments disclosed hereby. Flow controller 902 may be similar to flow controller 802, except that housing 904 is used in place of interface 808, housing 810, and biasing member 824a. Accordingly, housing 904 may function as a user interface, a housing, and a biasing member. One or more components of FIG. 9, or aspects thereof, may be incorporated into other embodiments of the present disclosure, or excluded from the disclosed embodiments, without departing from the scope of this disclosure. Further, one or more components of other embodiments of the present disclosure, or aspects thereof, may be incorporated into one or more components of FIG. 9, without departing from the scope of this disclosure. For example, primary control valve 804 with integrally formed seals 816a, 816b, 816c may be incorporated into flow controller 902 without departing from the scope of this disclosure. Embodiments are not limited in this context.

In the illustrated embodiment, the number of components is reduced by combining the interface, housing, and one biasing member into housing 904. The housing 904 may comprise a single elastomeric cap made from nitrile rubber, liquid silicone rubber, or a similar flexible material. For example, the housing 904 may have a Shore A value between 30-60. In various embodiments, the elastomeric cap can eliminate the need for two springs by acting as a biasing member and eliminating the outer spring of the assembly (see e.g., biasing member 824a of flow controller 802). In various such embodiments, the biasing member portion(s) of the elastomeric cap may have higher Shore values, such as a Shore A value between 50-100. The housing 904 would have cutouts at the bottom to snap onto the endoscope valve well preventing movement at the distal end of the cap and provide functionality of housing 810. The housing 904 may function as interface 808 because the elastomeric cap of housing 904 could be compressed and the elastomeric cap would flare out at the center. In many embodiments, the middle of the housing 904 may be made with a thinner wall thickness to encourage flaring out in that area. The housing 904 may function as the outer spring because the elastomeric cap would require some force to compress and the restorative force of the elastomeric cap material would cause the housing 904 to return to its original shape. In some embodiments, housing 904 may fold like an accordion to accomplish the spring function without flaring out, such as if space is a tight constraint.

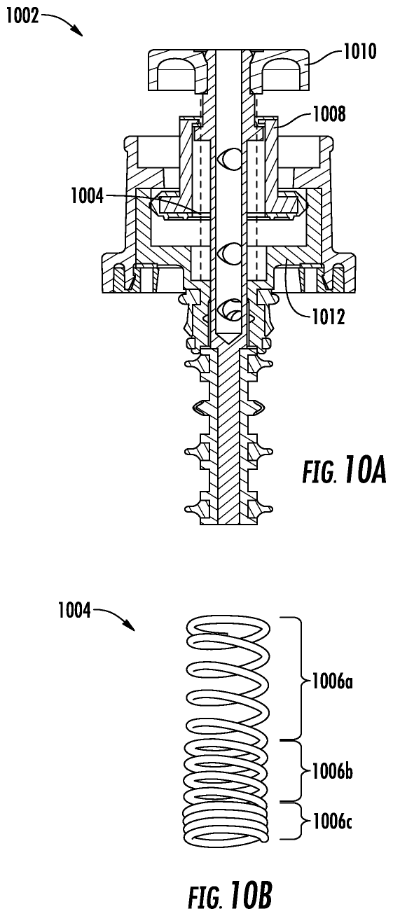

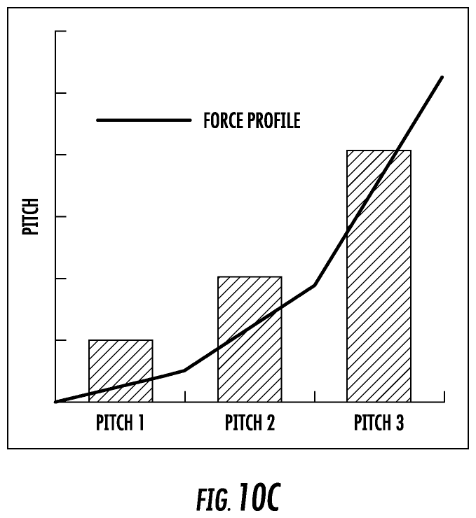

FIGS. 10A and 10B illustrate various aspects of a flow controller 1002 according to one or more embodiments disclosed hereby. More specifically, FIG. 10A illustrates a cross-sectional view of flow controller 1002 and FIG. 10B illustrates a variable rate spring 1004 of the flow controller 1002. Flow controller 1002 may be similar to flow controller 802, except that variable rate spring 1004 is used in place of biasing members 824a, 824b. Accordingly, variable rate spring 1004 may function as both biasing members 824a, 824b. Additionally, the variable rate spring 1004 may include a plurality of spring rates. As will be described in more detail below, in some embodiments, a middle portion of the variable rate spring 1004 may be coupled to the hat 1008 of flow controller 1002. One or more components of FIGS. 10A and 10B, or aspects thereof, may be incorporated into other embodiments of the present disclosure, or excluded from the disclosed embodiments, without departing from the scope of this disclosure. For example, variable rate spring 1004 may be incorporated into flow controller 802. Further, one or more components of other embodiments of the present disclosure, or aspects thereof, may be incorporated into one or more components of FIGS. 10A and 10B, without departing from the scope of this disclosure. For example, primary control valve 804 with integrally formed seals 816a, 816b, 816c may be incorporated into flow controller 1002 without departing from the scope of this disclosure. Embodiments are not limited in this context.

The variable rate spring 1004 may be disposed about a portion of the primary control valve of flow controller 1002. In many embodiments, the variable rate spring is disposed about a portion of the primary control valve between the top and the one or more seals. As previously mentioned, in some embodiments, a middle portion of the variable rate spring 1004 may be coupled to the hat 1008 of flow controller 1002. In such embodiments, the coupling between the hat 1008 and the variable rate spring 1004 may enable variable rate spring 1004 to provide a biasing force between the interface 1010 and hat 1008 with a first portion of the variable rate spring 1004 and a biasing force between the hat 1008 and the bowl 1012 with a second portion of the variable rate spring 1004.

The variable rate spring 1004 may provide a plurality of different spring rates 1006c, 1006b, 1006a. Accordingly, variable rate spring 1004 may replace an inner spring (e.g., biasing member 824b) with variable rate spring 1004. In several embodiments, variable rate spring 1004 may include a variable pitch spring or dual rate spring. In some embodiments, the variable rate spring 1004 may include two or more portions with different spring constants, such as between 1-10 N/mm. For example, a first portion of the spring may have a 1 N/mm spring constant and a second portion of the spring may have a 2 N/mm spring constant, resulting in the second portion being twice as hard to compress than the first portion. In many embodiments, the variable rate spring 1004 may have two or more sections of a spring that have different pitches (distance between adjacent coils). In the illustrated embodiment of FIG. 10B, variable rate spring 1004 includes first spring rate 1006c, second spring rate 1006b, and third 1006a. In various embodiments, spring rate 1006a may correspond to a portion of the variable rate spring 1004 that is the easiest to compress, spring rate 1006b may correspond to a portion of the variable rate spring 1004 with a smaller pitch distance that is more difficult to compress, and spring rate 1006c may correspond to a portion of the variable rate spring 1004 that is practically incompressible. Accordingly, in some embodiments, spring rate 1006c may be utilized as a spacer to properly position other portions of the variable rate spring 1004. In many embodiments, the varying spring rates may be realized using different helical pitches. In the illustrated embodiment, spring rate 1006a may correspond to a first pitch, spring rate 1006b may correspond to a second pitch, and spring rate 1006c may correspond to a third pitch. The principle behind variable pitch springs and how they require different forces at various stages of compression is illustrated in FIG. 10C.

The foregoing discussion has broad application and has been presented for purposes of illustration and description and is not intended to limit the disclosure to the form or forms disclosed hereby. It will be understood that various additions, modifications, and substitutions may be made to embodiments disclosed hereby without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated hereby. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

All of the devices and/or methods disclosed and claimed hereby can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method disclosed hereby without departing from the concept, spirit, and scope of the disclosure. All such similar substitutes and modifications apparent to those skilled in the art are deemed to be within the spirit, scope and concept of the disclosure as defined by the appended claims.

1. A medical device, comprising:
a primary control valve including a top, a bottom, one or more orifices, and a lumen in fluid communication with at least one orifice in the one or more orifices; and
one or more seals positioned between the top and bottom of the primary control valve, wherein the primary control valve and the one or more seals comprise a unitary structure.

2. The medical device of claim 1, wherein the primary control valve and the one or more seals are formed from an elastomer.

3. The medical device of claim 2, wherein the elastomer comprises nitrile rubber.

4. The medical device of claim 3, wherein the primary control valve comprises a first Shore value and the one or more seals comprise a second Shore value lower than the first Shore value.

5. The medical device of claim 1, further comprising a housing configured to couple with the primary control valve and a valve well.

6. The medical device of claim 5, wherein the housing is configured to provide a biasing force between the primary control valve and the valve well.

7. The medical device of claim 6, wherein the housing is formed from an elastomer.

8. The medical device of claim 1, further comprising a variable rate spring disposed about a portion of the primary control valve.

9. The medical device of claim 1, wherein the one or more seals are overmolded onto the primary control valve to form the unitary structure.

10. A method, comprising:
forming a primary control valve in a first stage of a molding process; and
forming one or more seals on the primary control valve in a second stage of the molding process to create a unitary structure having the primary control valve and the one or more seals.

11. The method of claim 10, further comprising allowing the primary control valve to begin to set between the first and second stages of the molding process.

12. The method of claim 10, wherein the primary control valve and the one or more seals are formed from an elastomer.

13. The method of claim 12, wherein the elastomer comprises nitrile rubber.

14. The method of claim 10, wherein the molding process comprises an injection molding process.

15. The method of claim 10, wherein the molding process comprises an overmolding process.

16. A medical device, comprising:
a primary control valve including a top, a bottom, one or more orifices, and a lumen in fluid communication with at least one orifice in the one or more orifices; and
one or more seals positioned between the top and bottom of the primary control valve, wherein the primary control valve and the one or more seals comprise a unitary structure; and
a housing configured to couple with the primary control valve and a valve well.

17. The medical device of claim 16, wherein the housing is configured to provide a biasing force between the primary control valve and the valve well.

18. The medical device of claim 16, wherein the housing is formed from an elastomer.

19. The medical device of claim 16, further comprising a variable rate spring disposed about a portion of the primary control valve.

20. The medical device of claim 16, wherein the one or more seals are overmolded onto the primary control valve to form the unitary structure.
